(19) United States
(12) Patent Application Publication
(10) Pub. No.: US 2024/0286234 A1
(43) Pub. Date: Aug. 29, 2024

(54) Spring Carrier

(71) Applicant: SANOFI WINTHROP INDUSTRIE, Antony (FR)

(72) Inventors: Philippe Chabert, Paris (FR); David Darras, Paris (FR)

(21) Appl. No.: 18/572,485

(22) PCT Filed: Jun. 23, 2022
(86) PCT No.: PCT/EP2022/067089

§ 371 Date: Dec. 20, 2023

(30) Foreign Application Priority Data
Jun. 24, 2021 (EP) 21315107.9

(51) Int. Cl. B23P 19/04

(52) U.S. Cl. CPC B23P 19/048

(57) ABSTRACT

A spring carrier for receiving, retaining, and discharging of a coil spring in a manufacturing process includes an elongate hollow body defining an inner cavity to receive the coil spring and an opening at a first proximal end of the hollow body for the insertion of the coil spring into the inner cavity and extraction of the coil spring from the inner cavity. The hollow body includes a second distal end opposite to the first proximal end. The hollow body includes a magnetic member to magnetically attract and retain the coil spring when the coil spring is located within the inner cavity.

### CROSS REFERENCE TO RELATED APPLICATIONS

The present application is the national stage entry of International Patent Application No. PCT/EP2022/067089, filed on Jun. 23, 2022, and claims priority to Application No. EP 21315107.9, filed on Jun. 24, 2021, the disclosures of which are incorporated herein by reference.

### TECHNICAL FIELD

The present disclosure relates to a device for carrying a spring, an apparatus including such a device, and a method of use of such a device and apparatus.

### BACKGROUND

Many devices require one or more springs and the method and apparatus for assembly of such devices requires accurate and repeated retrieval, movement and placement of such springs. Devices including one or more springs in their assembly include medicament injection devices. Such devices may include a spring to facilitate various functions of the device, including operation of a drug administration mechanism, or deployment of one or more safety features before, during or after a medicament delivery process.

Springs can readily become entangled if stored or conveyed together in a bulk manner, and separating springs when required to be assembled into a device being manufactured can be difficult and time consuming, and therefore inefficient and costly in terms of the manufacturing process. In high-volume manufacturing processes, errors in an assembly line or a need to pause a production line, for example due to a jam or fault in the machinery, is undesirable as it leads to lost production time, loss of productivity and product output, and impacts manufacturing and product costs.

In a manufacturing process of a product containing one or more springs, it is therefore desirable to provide a device that facilitates repeated and reliable retrieval, transport, and placement of such springs for use in such a process, and/or which may help protect and ensure spring integrity.

### SUMMARY

According to the present disclosure, there is provided a spring carrier for receiving, retaining and discharging of a coil spring in a manufacturing assembly process comprising an elongate hollow body defining an inner cavity configured to receive a coil spring, an opening at a first proximal end of the hollow body for the insertion and extraction of a coil spring into and from the inner cavity, the hollow body including a second distal end opposite to the first proximal end, wherein the hollow body includes a magnetic member configured to magnetically attract and retain a coil spring when located within the inner cavity.

The magnetic member may be disposed proximate to the second end of the hollow body. The magnetic member may be disposed proximate the first end of the hollow body, or disposed intermediate the first and second ends of the hollow body.

The magnetic member may be disposed within the inner cavity. The magnetic member may be disposed on the hollow body outside of the inner cavity. The magnetic member may be embedded within the hollow body.

The magnetic member may comprise a permanent magnet.

The magnetic member may comprise a magnetic material capable of induced magnetism.

The magnetic member may comprise an electromagnet. The spring carrier may further comprise at least one electrical contact electrically connected to the magnetic member for supply of electrical power to the electromagnet.

The hollow body may include a flange at the first proximal end of the hollow body and extending radially outwardly from the hollow body.

The flange may extend uninterrupted around the perimeter of the hollow body. The flange may be disposed at a proximal-most location of the hollow body.

The hollow body may be a cylindrical tube which is circular in cross-section.

The hollow body may be substantially uniform in cross-section dimension along its length.

The hollow body may be substantially rigid and not readily deformable from its cross-sectional shape.

The spring carrier may comprise an aperture at the second distal end of the hollow body.

The aperture at the second distal end of the hollow body may be of the same cross-sectional dimension as the cross-sectional dimension of the inner cavity.

The aperture at the second distal end of the hollow body may be of a smaller cross-sectional dimension than the cross-sectional dimension of the inner cavity.

The second distal end of the hollow body may be at least partially closed.

One or more protrusions may extend inwardly at least partially across an aperture at the second distal end of the hollow body.

The spring carrier may comprise at least one window in a side wall of the hollow body to allow a coil spring located within the spring carrier to be visible from outside the spring carrier through the window. The or each window may be formed in a side wall of the hollow body, and may be formed in a side wall of the hollow body in a location between the first proximal end and the second distal end of the hollow body.

The opening at the first proximal end of the hollow body may comprise a tapered region such that the first opening widens towards the first proximal end. The tapered region may extend at an angle with respect to the axis X-X of the spring carrier 10 of between 10 to 40 degrees, and may be between 15 to 35 degrees, and may be around 24 degrees.

The second distal end of the hollow body may include one or more projections extending inwardly from the hollow body. The or each projection may extend at least partially across an opening at the second distal end of the hollow body. The second distal end of the hollow body may include in inwardly-protruding lip extending at least partially around an opening at the second distal end. The second distal end of the hollow body may be partially or entirely closed by an end wall.

The spring carrier may comprise one or more orientation features configured for cooperation with corresponding orientation features on an apparatus with which the spring carrier may be used. The orientation feature(s) may allow the spring carrier to be accurately aligned in use. Such orientation feature(s) may comprise one or more recesses or slots in the flange. Such orientation feature(s) may comprise diametrically opposed slots in the flange.

The spring carrier may comprise one or more centering lugs projecting inwardly from an inside surface of a side wall of the hollow body. The centering lugs may project towards the central axis of the hollow body. The centering lugs may be equally spaced around the inside circumference of the side wall of the hollow body. The or each centering lug may be formed as ramp which increases in inward projecting distance in a direction towards the second distal end of the hollow body.

Also provided in the present disclosure is an apparatus comprising a spring carrier as described above, and an electromagnet configured for placement proximate the spring carrier and configured to generate a magnetic field to make the magnetic member an induced magnet.

Also provided in the present disclosure is an apparatus comprising a spring carrier as described above, and an airflow generator configured to generate a flow of air into the hollow body to facilitate extraction of a coil spring from the hollow body.

Also provided in the present disclosure is a manufacturing apparatus comprising a spring carrier as described above, and a spring extraction station configured to receive the spring carrier and locate the spring carrier whilst the coil spring is extracted from the spring carrier.

The spring carrier may include an airflow passage at the second distal end of the hollow body to allow the flow of air into and through the hollow body from the airflow generator.

The airflow generator and/or the airflow passage may be configured to direct the flow of air into the hollow body at an acute angle other than parallel relative to the central axis of the hollow body.

The airflow generator may comprise an air duct connectable or insertable to/into the second end of the hollow body.

Also provided in the present disclosure is an assembly system comprising an apparatus as described above and a coil spring manufacturing machine, wherein the coil spring manufacturing machine is configured to produce a coil spring, and the system further includes an insertion station arranged to feed the produced coil spring into the spring carrier.

The assembly system may further include the manufacturing apparatus comprising the extraction station described above.

The insertion station may comprise an electromagnet arranged to induce magnetism in the magnetic member.

The magnetic member may comprise an electromagnet and the insertion station may include an electrical power supply connectable to the magnetic member.

Also provided in the present disclosure is a method of manipulating a coil spring using a spring carrier for receiving, retaining and discharging of a coil spring in a manufacturing assembly process, the spring carrier comprising an elongate hollow body defining an inner cavity, an opening at a first proximal end of the hollow body, the hollow body including a second distal end opposite to the first proximal end, and a magnetic member provided proximate to the second end of the hollow body, the method comprising inserting the coil spring into the inner cavity through the opening at the first end of the hollow body and the magnetic member magnetically attracting and retaining the coil spring within the inner cavity.

Also provided in the present disclosure is a method of manipulating a coil spring using a spring carrier as described above, the method comprising inserting the coil spring into the inner cavity through the opening at the first end of the hollow body and the magnetic member magnetically attracting and retaining the coil spring within the inner cavity.

Also provided in the present disclosure is a method of manipulating a coil spring using a spring carrier for receiving, retaining and discharging of a coil spring in a manufacturing assembly process, the spring carrier comprising an elongate hollow body defining an inner cavity, an opening at a first proximal end of the hollow body, the hollow body including a second distal end opposite to the first proximal end, and a magnetic member provided proximate to the second end of the hollow body, the method comprising the magnetic member magnetically attracting and retaining the coil spring within the inner cavity, and extracting the coil spring from the hollow body by generating a flow of air into and through the hollow body towards the first proximal end of the hollow body.

Also provided in the present disclosure is a method of manipulating a coil spring using a spring carrier as described above, the method comprising the magnetic member magnetically attracting and retaining the coil spring within the inner cavity, and extracting the coil spring from the hollow body by generating a flow of air into and through the hollow body towards the first proximal end of the hollow body.

The method may comprise operating an electromagnet to create the magnetic force of attraction of the coil spring to the magnetic member.

The magnetic member may become an induced magnet by placement in the magnetic field of the electromagnet.

The magnetic member may comprise the electromagnet.

The spring carrier may comprise at least one window in a side wall of the hollow body, and the method may comprise detecting the presence or absence of a coil spring within the inner cavity of the hollow body by means of the or at least one of the windows. The detection of the presence or absence of a coil spring within the inner cavity of the hollow body by means of window(s) may comprise using a camera or optical sensor aligned with the window(s).

### BRIEF DESCRIPTION OF THE FIGURES

Embodiments will now be described, by way of example only, with reference to the accompanying drawings, in which:

FIG. 1 is a perspective view of a spring carrier of an embodiment of the present disclosure;

FIG. 2 is another perspective view of the spring carrier of FIG. 1;

FIG. 3 is an enlarged cut away perspective view of a region at a second end of the spring carrier of FIGS. 1 and 2;

FIGS. 4A-4B show a sequence of steps of use of the spring carrier of FIGS. 1-3 during insertion of a coil spring into the spring carrier;

FIGS. 5A-5B show a sequence of steps of use of the spring carrier of FIGS. 1-4B during extraction of a coil spring from the spring carrier;

FIG. 6 is a perspective view of the spring carrier of FIGS. 1-5B in combination with a receiving portion of an apparatus of an embodiment;

FIG. 7A is an enlarged perspective view of an end portion of a spring carrier of another embodiment;

FIG. 7B is a cross-sectional view of the end portion of the spring carrier of FIG. 7A;

FIG. 8A is an enlarged perspective view of an end portion of a spring carrier of another embodiment;

FIG. 8B is a cross-sectional view of the end portion of the spring carrier of FIG. 8A;

FIG. 9 is a cross-sectional side view of a spring carrier of another embodiment;

FIG. 10 is a cross-sectional side view of a spring carrier of another embodiment;

FIG. 11 is an enlarged perspective view of an end portion of a spring carrier of another embodiment;

FIG. 12 is an enlarged perspective view of an end portion of a spring carrier of another embodiment;

FIG. 13 is a perspective view of a spring carrier of another embodiment of the present disclosure;

FIG. 14 is a perspective view of a spring carrier of another embodiment of the present disclosure;

FIG. 15 is a perspective view of a spring carrier of another embodiment of the present disclosure;

FIG. 16 is a perspective view of a spring carrier of another embodiment of the disclosure;

FIG. 17 is a cross-sectional view of a portion of the spring carrier of FIG. 16; and

FIG. 18 is a schematic view of an assembly system of an embodiment of the disclosure.

### DETAILED DESCRIPTION

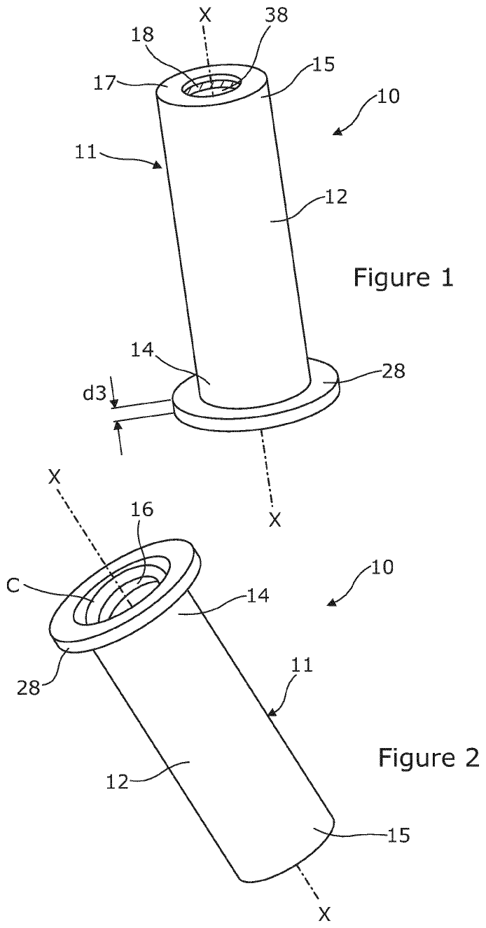

FIGS. 1 to 3 show a spring carrier 10 of an embodiment of the disclosure, and which comprises a hollow body 11 having a side wall 12 formed as a tube and defining an inner cavity 13. The hollow body 11 includes opposite first, proximal and second, distal ends 14, 15. The hollow body 11 is circular in cross-section and comprises a central axis X-X. A first opening 16 is provided at the first proximal end 14 to allow access into the inner cavity 13. The second distal end 15 is provided with an end wall 17 which partially closes the second end 15 of the hollow body 11. The end wall 17 includes an aperture 38 communicating with the inner cavity 13 from the second end 15.

The spring carrier 10 includes a magnetic element or member 18 proximate the second end 15 of the hollow body 11. In the exemplary embodiment shown, the magnetic member 18 is disposed within the inner cavity 13 on an inside surface of the end wall 17, and is ring-shaped to surround the aperture 38 in the end wall 17. In the exemplary embodiment, the magnetic member 18 comprises a permanent magnet.

A flange 28 is provided on the outer surface of the hollow body 11 and extends radially outwardly in a direction perpendicular to the central axis X-X. In the exemplary embodiment shown, the flange 28 is located at the distal-most region of the first end 14 of the hollow body 11.

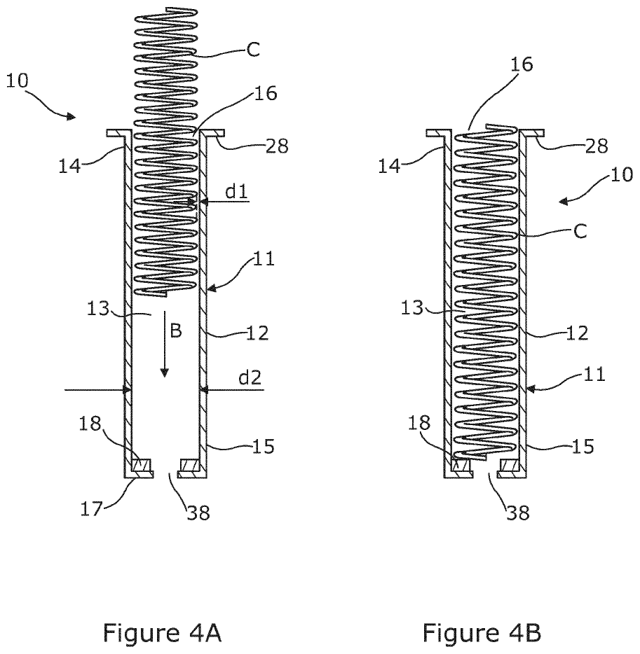

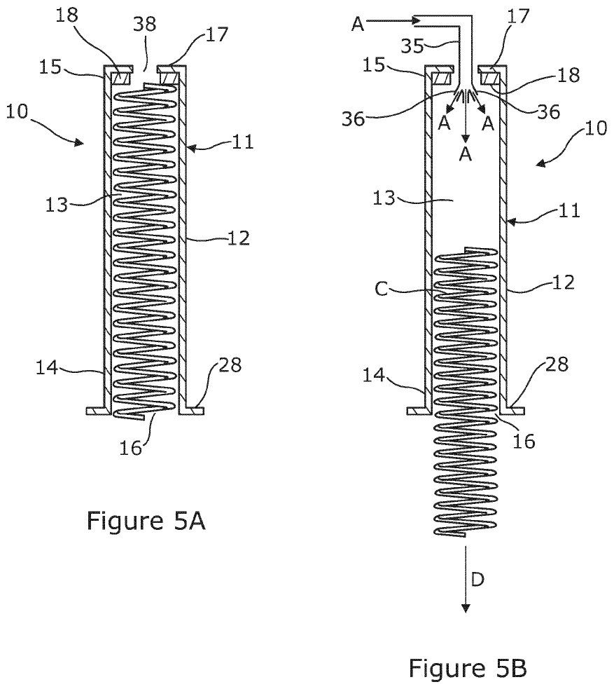

In use during a manufacturing and assembly process, the spring carrier 10 is used to receive, retain, convey, and discharge a coil spring C. Such a manufacturing process may include, for example, a method of manufacturing a medicament delivery device in which a coil spring C may be included as a biasing member to actuate a drug administration mechanism or to actuate a needle safety mechanism after a drug has been administered. Use of the spring carrier 10 will now be described with reference to FIGS. 4A-4B and FIGS. 5A-5B.

The spring carrier 10 is intended to be used as part of an apparatus of the present disclosure. Such apparatus may comprise a spring carrier apparatus that may comprise part of an assembly system or apparatus for a medical device, and may comprise part of an assembly and/or manufacturing apparatus/system for a medicament injection device. However, the disclosure is not intended to be limited to the medical device field and is applicable to any technical field in which one or more springs may be required to be handled and conveyed.

FIGS. 4A-4B show method steps of insertion of a coil spring C into the spring carrier 10. In the first step shown in FIG. 4A, the spring carrier 10 is arranged with first opening 16 in the first end uppermost, and a coil spring C is introduced into the empty inner cavity 13. This coil spring C may be placed into the inner cavity 13 or dropped into the inner cavity 13 so that it falls under its own weight into the inner cavity 13. The coil spring C is inserted into the inner cavity 13 in the direction shown by arrow B until it comes to rest against the magnetic member 18 at the end wall 17 of the hollow body 11.

The coil spring C is made of a magnetically-attractable metal, for instance a ferro-magnetic material, such as steel. As such, the coil spring C is attracted to and magnetically held against the magnetic member 18. The magnetic member 18 is capable of providing an attractive force on the coil spring C that is greater than the weight of the coil spring C. The coil spring C is thereby securely retained within the spring carrier 10 and can be conveyed within the spring carrier 10 to a location and manufacturing/assembly apparatus where the coil spring C is to be utilized.

The extraction process of the coil spring C from the spring carrier 10 is described with reference to FIGS. 5A-5B. Before the extraction process starts, and at a preceding step in the assembly or manufacturing process including the coil spring C, the spring carrier 10 is inverted from the orientation shown in the insertion method steps of FIGS. 4A and 4B, so that the spring carrier 10 is oriented with the first end 14 lowermost and the second end 15 uppermost. The spring carrier 10 is also positioned directly above a location where the coil spring C is to be deposited for the respective assembly/manufacturing process. For instance, the spring carrier 10 may be aligned vertically for the extraction process. This may help the coil spring C be extracted consistently and squarely from the spring carrier 10, that is, in a direction aligned with the central axis X-X of the hollow body 11.

In the first step shown in FIG. 5A, the spring carrier 10 is in its inverted position with the first end 14 lowermost, and the coil spring C is retained within the inner cavity 13 by magnetic attraction to the magnetic member 18.

In the second step, the coil spring C is extracted from the spring carrier 10. This may be achieved by various means within the scope of the disclosure. To extract the coil spring C, a sufficient force needs to be exerted on the coil spring C in an extraction direction shown by arrow D in FIG. 5B to overcome the attraction force between the magnetic member 18 and the coil spring C. Such extraction force may be provided by pulling means (not shown) engaging the coil spring C and pulling the coil spring C out of the spring carrier 10, or the apparatus may include impact means (not shown) configured to provide an impact force on the spring carrier 10 to jolt the coil spring C out of the spring carrier 10.

In the exemplary embodiment shown in FIG. 5B, means for facilitating extraction of the coil spring C from the spring carrier 10 comprises an airflow source or air jet A to generate a flow of air through the inner cavity 13 to exert an airflow force on the coil spring C which is sufficient to blow the coil spring C out of magnetic engagement with the magnetic member 18 and out of the spring carrier 10. The airflow source A may comprise an air duct or air passage 35 which may be disposed through or connected to the second end 15 of the hollow body 11. Such air passage 35 may be insertable into or disposed through the aperture 38 in the end wall 17 of the spring carrier 10. The air passage 35 may be connected or connectable to a source of pressurized air A. In use, the air source A may be connected, or turned on to send a flow of air (shown by arrows A in FIG. 5B) through the air passage 35 and into the inner cavity 13 of the hollow body 11. The air flow A may then impinge upon the coil spring C and force the coil spring C out of magnetic engagement with the magnetic member 18 and out of the spring carrier 10.

A plurality of air passages 35 may be provided, and/or a plurality of air passage outlets 36 may be provided. The air passage(s) 35 and/or air flow outlets 36 may be aligned substantially parallel with the central axis X-X of the hollow body 11 in use. In addition, or alternatively, one or more air flow outlets 36 and/or air flow passages 35 may be oriented at an angle with respect to the central axis X-X of the hollow body 11 in use. In the latter case, the angled air flow outlets 36/passages 35 may encourage the air flow to impinge on the coils of the coil spring C to encourage expulsion of the coil spring C from the spring carrier 10. In an embodiment with angled airflow passages 35 or outlets 36 to generate a flow of air at an angle with respect to the central axis X-X of the hollow body 11, such angle may be acute other than perpendicular or parallel to the central axis X-X, and may for instance be between 0-45 degrees, and may be between 5-30 degrees. In an embodiment in which a central axial air flow passage 35/outlet 36 is provided, turbulence of air flow through the coil spring may still cause sufficient impinging of the air flow on the coils of the coil spring C to achieve disengagement of the coil spring C from the magnetic member 18 and expulsion of the coil spring C from the spring carrier 10.

The air passage(s) 35 may be a separate component of an apparatus to the spring carrier 10, or may comprise a component connected to, or formed integrally with, the spring carrier 10, as will be explained in more detail below.

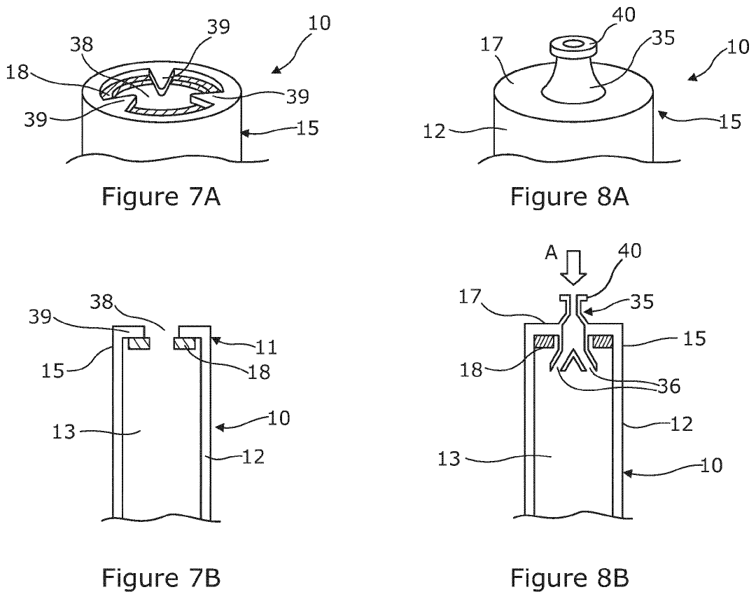

Referring to FIGS. 7A to 8B, alternative variations of spring carrier 10 are illustrated. Like features retain the same reference numerals and a detailed description thereof will not be repeated. In the embodiment of FIGS. 7A and 7B, the second end 15 of the hollow body 11 is not partially closed by an end wall 17 but still includes an opening or aperture 38. Around the perimeter of the aperture 38 are a plurality of protrusions 39 extending radially inwardly. In use, the protrusions 39 may serve to support the magnetic member 18. For example, the magnetic member 18 may be bonded, mechanically fastened, or otherwise secured to the protrusions 39. Alternatively, or in addition, the protrusions 39 may acts as spring stops for the coil spring C to rest against when being inserted into the spring carrier 10 (as described above with reference to FIGS. 4A and 4B). This may be the case in embodiments of the disclosure in which the magnetic member 18 may be disposed on an outside of the hollow body 11 (described in more detail below). The aperture 38 may be used as an access aperture through which an airflow duct or air passage 35 may extend in variants of the disclosure in which an airflow source A is used to assist extraction of the coil spring C from the spring carrier 10.

In the embodiment of FIGS. 8A and 8B, the end wall 17 includes an airflow duct 35 extending through the end wall 17. The airflow duct 35 may be a separate component secured to the end wall 17, such as by bonding, mechanical fastening, welding or other known means. Alternatively, the airflow duct 35 may be formed integrally with the end wall 17 and the hollow body 11. The airflow duct 35 includes a plurality of outlets 36 within the inner cavity 13. The outlets 36 are oriented at an acute angle other than perpendicular or parallel to the central axis X-X of the hollow body. This may help provide the advantages mentioned above. The angles of the outlet orientation may be any of those described above. However, in alternative embodiments, there may be only one outlet 36 or more than two outlets 36, and/or the or each outlet may be oriented substantially parallel with the central axis X-X of the hollow body 11. The airflow duct 35 includes an inlet 40. In use, pressurized airflow A may be fed into the airflow duct 35 through the inlet 40. The inlet may be configured with a connection for coupling the airflow duct 35 to an airflow source A.

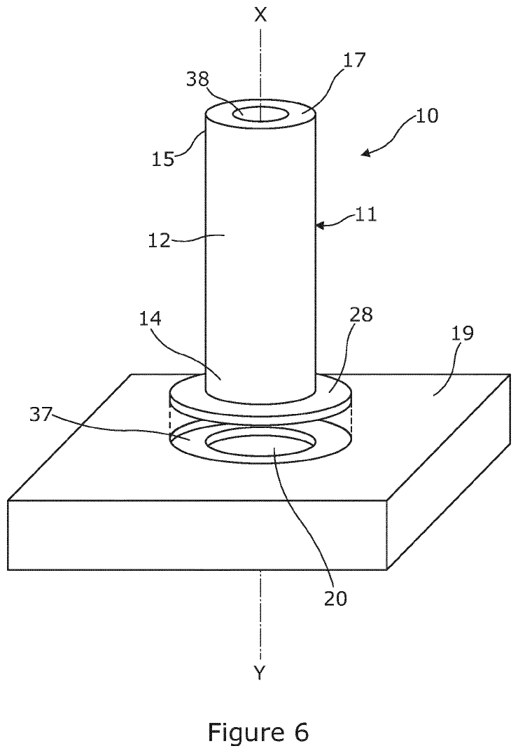

In both the insertion process and extraction process, the spring carrier 10 may be accurately aligned with the location from and to which the coil spring C is to be inserted/extracted to allow the coil spring C to be effectively conveyed as desired and not snag upon extraction from the spring carrier 10, for example on an apparatus into which the coil spring C is to be discharged. In this way, manufacturing errors and/or production stoppages to correct the errors can be reduced or avoided. FIG. 6 shows an embodiment of an apparatus of the disclosure including the spring carrier 10 described above, and a receiving portion 19 which is configured to receive a coil spring C extracted from the spring carrier 10. The receiving portion 19 includes a receiving hole 20 into which the coil spring C may be discharged, and the receiving hole having a central axis Y-Y. The receiving portion 19 is configured to assist accurate spring carrier 10 alignment. The receiving portion 19 includes a recess 37 which is shaped to correspond to, and receive, the flange 28 of the spring carrier 10. This location feature can help towards ensuring the central axis X-X of the hollow body 11 is coaxial with the central axis Y-Y of the receiving hole 20, and that the coil spring C can be thereby accurately extracted from the spring carrier.

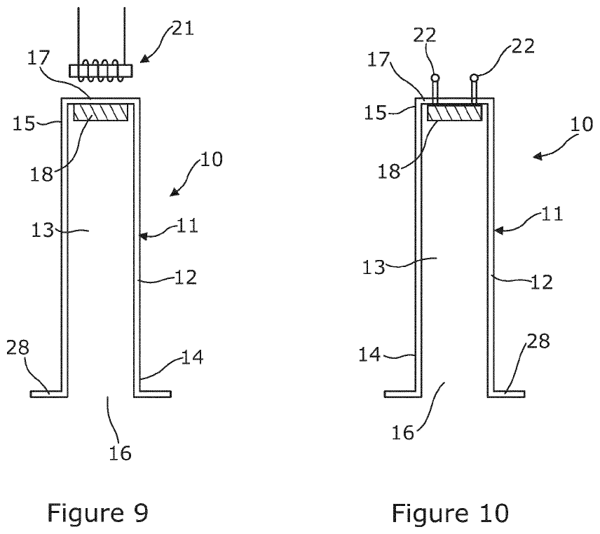

In the exemplary embodiments described above, the magnetic member 18 is described as a permanent magnet. However, the disclosure is not intended to be limited to such a configuration of spring carrier 10. Another alternative embodiment is shown in FIG. 9, and like features retain the same reference numerals and a detailed description thereof will not be repeated. The spring carrier 10 comprises an end wall 17 although there is no aperture 38 provided in the end wall 17. A magnetic member 18 is provided within the inner cavity 13 and on an inner surface of the end wall 17. The magnetic member 18 may take any suitable shape, but in the exemplary embodiment shown, comprises a disc-shaped component. A difference in this embodiment is that the magnetic member 18 does not comprise a permanent magnet, but instead is a magnetisable material. The spring carrier 10 may be used with an electromagnet 21 is disposed proximate the second end 15 of the spring carrier 10. When the coil spring C is inserted into the inner cavity 13, an electromagnet 21 may be disposed proximate the end wall 17 and powered to induce a magnetic effect in the magnetic member 18. This causes the coil spring C to be attracted to the magnetic member 18 and thereby securely retained within the inner cavity 13 of the spring carrier 10. In use of the embodiment, in a step equivalent to that shown in FIGS. 5A/5B, when the coil spring C is to be extracted from the spring carrier 10, the electromagnet 21 is unpowered, which stops the induced magnetic effect in the magnetic member 18, and so the coil spring C is no longer retained within the spring carrier 10, and so can be extracted therefrom in any manner mentioned above. It will be appreciated that a variant of this embodiment may include an aperture 38 as described previously in order that an airflow source A may be used to assist extraction of the coil spring C from the spring carrier 10.

A further alternative embodiment is described below and illustrated in FIG. 10. Like features retain the same reference numerals and a detailed description thereof will not be repeated. The spring carrier 10 comprises an end wall 17 although there is no aperture 38 provided in the end wall 17. A magnetic member 18 is provided which, in the exemplary embodiment shown in FIG. 10, is disposed within the inner cavity 13 and on an inner surface of the end wall 17. However, the magnetic member 18 may alternatively be disposed outside the inner cavity 13, or may be embedded within the wall of the hollow body 11. The magnetic member 18 may take any suitable shape. A difference in this embodiment is that the magnetic member 18 comprises an electromagnet which may be powered to create a magnetic field to cause the coil spring C to be attracted to the magnetic member 18 and thereby securely retained within the inner cavity 13 of the spring carrier 10. The magnetic member 18 comprises electrical terminals 22 for connection to a source of electrical power to power the electromagnet. The terminals 22 may be disposed on the outside of the spring carrier 10 and may be connectable to a source of electrical power provided on an apparatus with which the spring carrier 10 is to be utilized, particularly an apparatus which conveys the spring carrier 10 (and coil spring C retained therein) to an extraction station at which the coil spring C is to be extracted from the spring carrier 10.

In use of the embodiment of FIG. 10, in a step equivalent to that shown in FIGS. 5A/5B, when the coil spring C is to be extracted from the spring carrier 10, the electromagnet is unpowered, which stops the magnetic effect and so the coil spring C is no longer retained within the spring carrier 10, and so can be extracted therefrom in any manner mentioned above. It will be appreciated that a variant of this embodiment may include an aperture 38 as described previously in order that an airflow source A may be used to assist extraction of the coil spring C from the spring carrier 10.

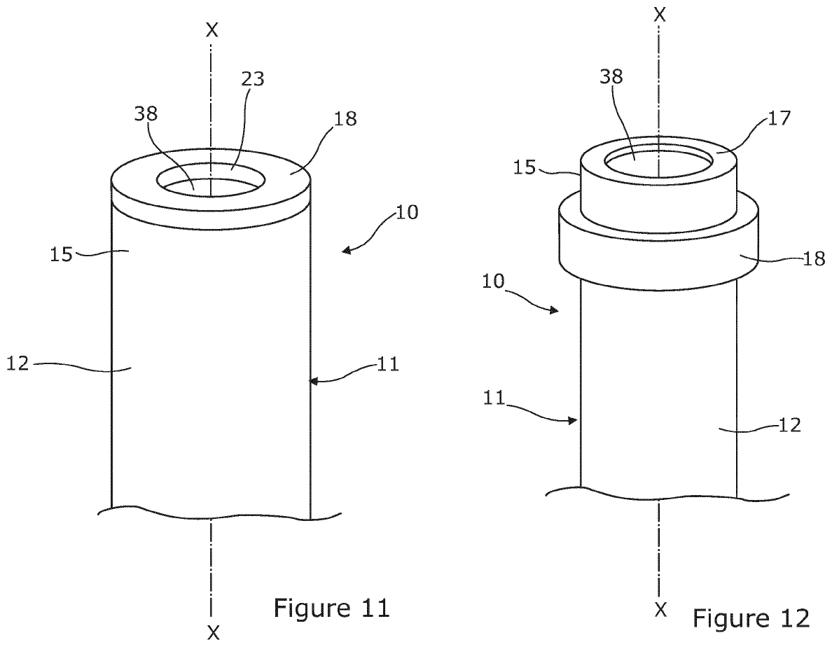

Further embodiments of spring carriers intended to be encompassed within the scope of the present disclosure are shown in FIGS. 11 and 12. Like features retain the same reference numerals and a detailed description thereof will not be repeated. The spring carriers 10 each comprise an end wall 17 having an aperture 38 provided in the end wall 17. A difference with the embodiments of FIGS. 11 and 12 are that the magnetic member 18 is provided outside of the inner cavity 13 instead of within the inner cavity 13 as in the previously-described embodiments. In the embodiment of FIG. 11, the magnetic member 18 comprises a ring shaped component comprising a permanent magnet, and a central aperture 23 of the ring-shaped magnetic member 18 is aligned with the aperture 38 in the end wall 17. The embodiment of spring carrier 10 of FIG. 12 also includes a magnetic member 18 that is a permanent magnet and which is provided outside of the inner cavity 13. However, in the embodiment of FIG. 12, the magnetic member 18 is not disposed on the end wall 17 but circumferentially around the side wall 12 of the hollow body 11 proximate the second end 15 thereof. In use, operation of the spring carrier 10 of the embodiments of FIGS. 11 and 12 is the same as that of the spring carrier 10 comprising a permanent magnet as a magnetic member 18 and described previously. In particular, in the coil spring C extraction process, the coil spring C may be extracted by any means described above, including providing an extraction force by pulling means (not shown) engaging the coil spring C and pulling the coil spring C out of the spring carrier 10, or impact means (not shown) configured to provide an impact force on the spring carrier 10 to jolt the coil spring C out of the spring carrier 10, or an airflow source or air jet A to generate a flow of air through the inner cavity 13 to exert an airflow force on the coil spring C sufficient to blow the coil spring C out of magnetic engagement with the magnetic member 18 and out of the spring carrier 10.

In the embodiments of FIGS. 11 and 12, the external magnetic members may be secured to the hollow body 11 by any suitable means, for example by being bonded to the hollow body 11, mechanically fastened, co-moulded with the hollow body or embedded within the hollow body 11.

In variants of the embodiments of spring carriers 10 shown in FIGS. 11 and 12, the magnetic members 18 may not be permanent magnets, but may comprise a magnetisable material, that is, a material in which a magnetic effect may be induced. Such material may be as described previously. The magnetic effect may be induced by placement in proximity to an electromagnet as described previously. The induced magnetic effect is sufficiently strong to attract and retain a coil spring C within the spring carrier 10 without the coil spring C being in direct contact with the magnetic member. The magnetic effect will need to act on the coil spring C through the material of the hollow body 11, either at the end wall 17 or a region of the side wall 12 proximate the second end 15 of the hollow body.

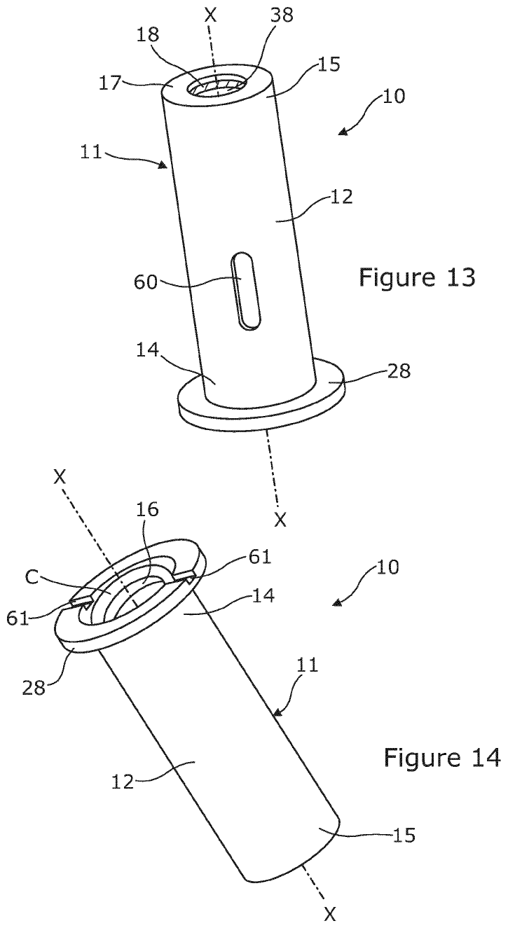

The spring carrier 10, and apparatus comprising the spring carrier 10 and receiving portion 19 of the apparatus, may be part of a larger assembly system or apparatus for manufacturing devices which include one or more coil springs C. Such system may comprise a plurality of assembly machines or stations. Such assembly machines/stations may be configured as an inline process and as two or more separate processes. An exemplary assembly system 50 is shown schematically in FIG. 13. The assembly system 50 includes a coil spring making system, generally designated 51. The coil spring making system 51 may include a coiling station 52 which creates the coil spring C, a heating station 53 where the coiled spring is heated to temper the material of the spring. The heated coiled spring is then fed to a cooling station 54 to cool the coiled spring. Thereafter, a conveyor 55 transfers the cooled coiled spring C to an insertion station 56. At the insertion station 56, the spring carrier 10 is operated as described above to insert the coil spring C into the spring carrier 10. The insertion station 56 may comprise an electromagnet 21 or electrical power for connection to terminals 22 of an electromagnet magnetic member 18 in the spring carrier 10 of respective embodiments described above. The spring carrier 10 with coil spring retained therein is conveyed to an extraction station 57. At the extraction station 57, spring carrier 10 is operated in any of the ways described above to extract the coil spring C from the spring carrier 10 for use in subsequent device assembly steps in which the coil spring C is utilized. The extraction station 57 may include an impactor to jolt the spring carrier 10 to release the coil spring C, or a mechanical extractor to grip and remove the coil spring C from the spring carrier 10.

FIG. 13 shows a spring carrier 10 of another embodiment of the disclosure, and like features retain the same reference numerals and detailed description thereof will not be repeated. A difference with the embodiment of FIG. 13 is that a window or cut-out region 60 is provided in and extending through the side wall 12 of the hollow body 11. This enables the interior of the hollow body 11 to be viewed from the outside of the spring carrier 10. Particularly, this enables a coil spring C to be seen when received within the spring carrier 10. This may benefit use of the spring carrier 10 in a manufacturing process. For example, in a quality control or performance monitoring process, the presence of a coil spring C within the spring carrier 10 may be checked for each device being produced. For example, an optical sensor or camera may check for the presence of a coil spring C within the spring carrier 10 and may operate using the window 60 to make such checks. For example, if it is detected that a coil spring C is absent from the spring carrier 10 due to an insertion fault elsewhere in the manufacturing process, the device being produced will likely not function correctly without the coil spring C and so can be automatically rejected from the production line. One window 60 may be provided, or a plurality of windows may be provided, and maybe disposed in any suitable location on the side wall 12 of the spring carrier 10. The windows 60 also mean that less material is required to manufacture each spring carrier 10, which may reduce cost of manufacture and/or may also reduce the weight of the spring carrier which may be beneficial in the device manufacturing processes in which the spring carrier is to be used. The feature of one or more windows 60 may be applicable to and provided with any embodiment of the disclosure disclosed herein.

FIG. 14 shows a spring carrier 10 of another embodiment, similar to the previously-described embodiments, and like features retain the same reference numerals and detailed description thereof will not be repeated. A difference with the embodiment of FIG. 14 is that the flange 28 includes orientation features 61. In the exemplary embodiment shown, the orientation features 61 comprise a pair of radial slots formed into the surface of the flange 28 facing in the direction of the first end 14. Such orientation features 61 may facilitate correct rotational positioning of the spring carrier 10 about its central axis X-X which may be beneficial for function of the spring carrier 10 in use, for example for insertion or extraction of a coil spring C. Furthermore, such orientation feature 61 may be used in conjunction with the window 60 during a manufacturing process. For example, an optical sensor or camera used to detect the presence of a coil spring C within the spring carrier 10 may be located in a certain position on a manufacturing apparatus/system or assembly line, and so require correct orientation of the spring carrier 10 to align the window 60 with the optical sensor or camera. The orientation features 61 may cooperate with corresponding features (not shown) such as projections which may be received in the slots of the orientation features 61 to ensure correct positioning of the spring carrier 10 in use. Such orientation feature(s) 61 may be applicable to and provided with any embodiment of the disclosure disclosed herein.

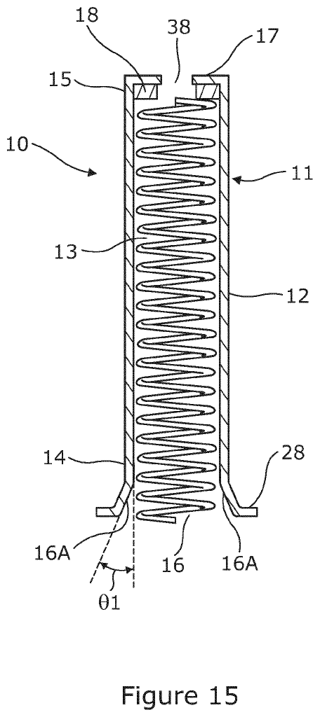

FIG. 15 shows a spring carrier 10 of another embodiment, similar to the previously-described embodiments, and like features retain the same reference numerals and detailed description thereof will not be repeated. FIG. 15 is a view similar to that of FIG. 5A of the previously-described embodiment, although a difference with the embodiment of FIG. 15 is that the first opening 16 at the first end 14 of the spring carrier includes a tapered region 16A such that the first opening 16 widens in a direction towards the first proximal end. This may help guide the coil spring C into the first opening 16 during the insertion step described above. Such feature may be applicable to and provided with any embodiment of the disclosure disclosed herein. As can be seen from FIG. 15, the tapered region 16A of the first opening 16 extends at an angle θ1 with respect to the axis X-X of the spring carrier 10. The angle θ1 may vary within the scope of the disclosure but may be between 10 to 40 degrees, and may be between 15 to 35 degrees, and may be around 24 degrees.

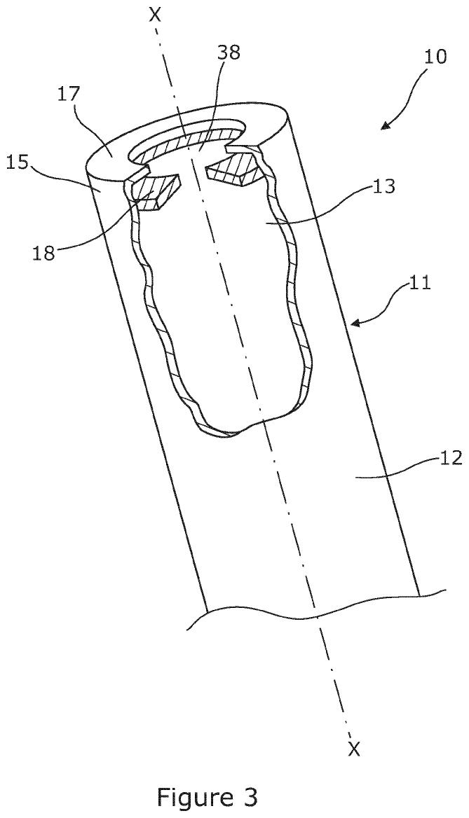

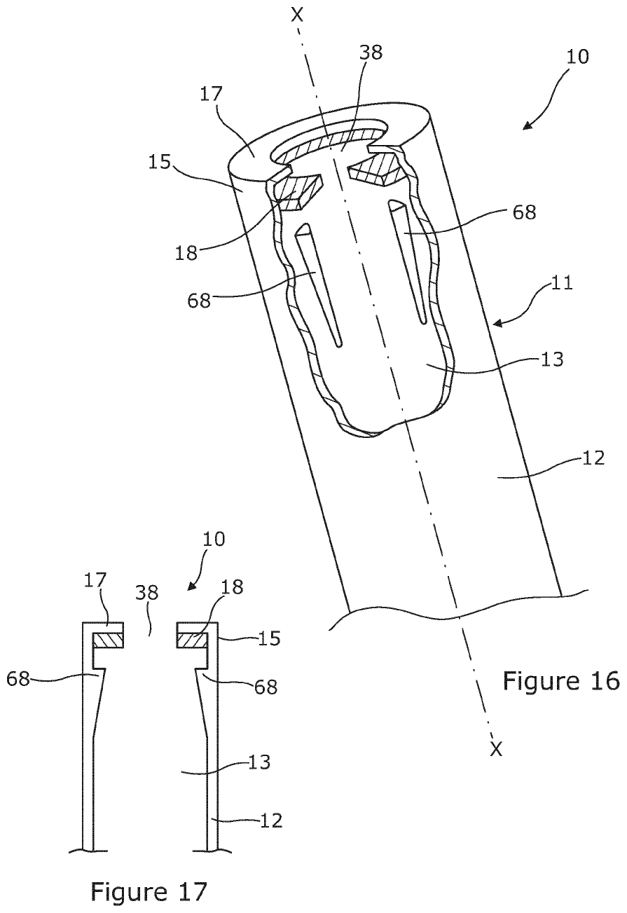

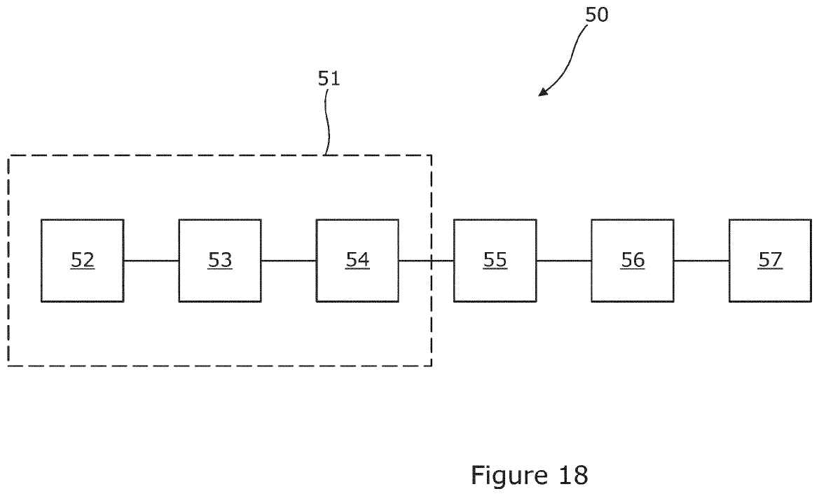

FIGS. 16 and 17 show a spring carrier 10 of another embodiment of the disclosure, similar to the spring carrier of FIGS. 1-3, and like features retain the same reference numerals and detailed description thereof will not be repeated. A difference in the spring carrier of FIGS. 16 and 17 is that the inner surface of the side wall 12 of the hollow body 11 includes a plurality of centering lugs 68 which project inwardly towards the central axis X-X of the hollow body 11. FIG. 16 is a view similar to that of FIG. 3 of the previously described embodiment and so shows a portion of the side wall 12 cut-away to enable these centering lugs 68 to be illustrated. In the embodiment shown, four centering lugs 68 are provided. However, more than four or fewer than four may be provided, and the centering lugs 68 may optionally be equally spaced around the inside circumference of the side wall 12.

The centering lugs 68 are formed as ramps with a curved surface and increase in the distance they project inwardly as the centering lug 68 extends towards the second distal end 15 of the spring carrier 10. In use, the centering lugs 68 serve to contact and center a coil spring C held within the spring carrier 10 so that the coil spring C is accurately retained centrally within the spring carrier 10. The centering lugs 68 may compensate for any tolerance between the outer diameter of the coil spring C and internal diameter of the inner cavity 13 to reduce play between the coil spring C and spring carrier 10. This may help ensure the coil spring C is accurately located during insertion of the coil spring C into the spring carrier 10, to help ensure the coil spring C can be securely engaged by the magnetic member 18. This may help avoid accidental or premature spring extraction during transportation of the spring carrier 10 or during a manufacturing process in which the coil spring is required to be accurately extracted and positioned into a device being manufactured. The may help prevent manufacturing errors and/or stoppages. The feature of the centering lugs 68 may optionally be applicable to and provided with any embodiment of the disclosure described herein.

In any of the embodiment disclosed herein, the spring carrier 10 may comprise a total length in a direction of the axis X-X of between 50 mm to 90 mm, and may be between 60 mm to 80 mm, and may be around 70.5 mm or around 73.5 mm.

In any of the embodiments disclosed herein, the flange 28 may comprise a height d3 in a direction of the axis X-X of between 1 mm to 5 mm, and may be between 2 mm to 4 mm, and may be around 3 mm.

In any of the embodiments disclosed herein, when comprising one or more windows 60, the or at least one of the windows 60 may comprise a length in a direction of the axis X-X of between 5 mm to 25 mm, and may be between 10 mm to 20 mm, and may be around 15 mm.

In embodiments described herein, the magnetic member 18 is described as being disposed proximate the second, distal end 15 of the hollow body 11. However, the disclosure is not intended to be limited to such configuration and in other embodiments intended within the scope of the present disclosure, the magnetic member 18 may be disposed proximate the first, proximal end 14 of the hollow body 11, or may be disposed intermediate the first and second ends 14, 15 of the hollow body 11.

The hollow body 11 is shown and described as being configured as a cylindrical tube which is circular in cross-section. This allows to closely contain coil springs C of conventional circular form. This also may facilitate ease of insertion of the coil springs C, and alignment of the spring carrier 10 for extraction of the coil spring C, as no specific rotational orientation about the central axis X-X is required for correct positioning of the spring carrier 10 in use. However, the disclosure is not intended to be limited to such a configuration of spring carrier, and other dimensions and cross-sectional shapes are possible, such as oval, triangular or square, or other polygons.

The hollow body 11 is shown and described as being of a substantially constant cross-section along its length from the first end 14 to the second, opposite end 15. This may facilitate ease and cost of manufacture and of manipulation in assembly or manufacturing processes in which the spring carrier 10 is to be utilized. However, the disclosure is not intended to be limited to such a configuration and in alternative embodiments, the spring carrier 10 may vary in cross-sectional dimension along its length. For example, the cross-section may be circular of different diameters along the length of the spring carrier, and/or the cross-section may be shaped other than circular along part of the length of the spring carrier. For example, the internal diameter may be larger in the region of the first end 14 through which the coil spring C is inserted and extracted, than in the region of the second end 15. This may further help accurately guide the coil spring C into the spring carrier 10. This may be in addition to assistance from a tapered region 16A if provided. This may also allow the coil spring C to be more closely confined in the region of the second end 15 of the spring carrier 10. However, the opposite may be the case within the scope of the disclosure and the internal diameter at the first end 14 may be smaller than the internal diameter at the second end 15 such that the inner cavity 13 is slightly narrower in the region of the first end 14 of the spring carrier 10.

In an exemplary embodiment in which the internal diameter is substantially uniform along the length of the hollow body 11, the internal diameter may be between 7 mm to 14 mm, and may be between 8 mm to 13 mm, and may be between 9 mm to 12 m, and may be between 10 mm to 11 mm, and may be around 10.5 mm or around 11.5 mm.

In an exemplary embodiment in which the internal diameter is not uniform along the length of the hollow body, the internal diameter at one end of the hollow body may be between 9 mm to 14 mm, and may be between 10 mm to 13 mm, and may be between 11 mm to 12 m, and may be around 11.5 mm. The internal diameter at the other end of the hollow body may be between 8 mm to 13 mm, and may be between 9 mm to 12 mm, and may be between 10 mm to 11 m, and may be around 10.5 mm.

It will be appreciated that in embodiments of the disclosure described herein, for the coil spring C to be retained within the spring carrier 10 during transit, movement, and changes in orientation and inversion of the spring carrier 10 in use, the magnetic member 18 is capable of exerting a force of magnetic attraction upon the coil spring C greater than the weight of the coil spring C. Additionally, the force of magnetic attraction the magnetic member 18 exerts on the coil spring C may exceed the weight of the coil spring C by a force margin to allow the coil spring C to remain securely retained within the spring carrier 10 during movement and handling of the spring carrier 10 in a manufacturing process and within manufacturing apparatus and system. The force margin may be selected to achieve a secure retention of a coil spring C within the spring carrier 10, yet enable reliable extraction of the coil spring C, either by knocking or other mechanical agitation, force of airflow as described above, or other mechanical extraction during a manufacturing process. Such force margin may be between 5-50%, for instance between 10-30%. The minimum force margin may be 5%.

Various materials may be selected from which the spring carrier 10 is formed, which includes plastics and metals, and may include various polymers, including Polypropylene, Polyester, Copolyester, Polyamide, Acrylo-Butadiene-Styrene (ABS) or Polycarbonate. The spring carrier may further be formed from Polycarbonate, and may comprise recycled Polycarbonate.

The hollow body 11 of the spring carrier 10 is shown and described as a single moulded component, that is, a single integral component. This may provide advantages of ease and reduced cost of manufacture. The magnetic member 18 may be bonded or mechanically fastened in the spring carrier 10, or may be co-molded with hollow body 11, or may be embedded within hollow body 11.

The side wall 12 and end wall 17 of the hollow body 11 are for instance of a dimension to provide sufficient structural strength during use, but also minimize excess use of material and maintain light-weight for ease of handling and cost of manufacture. The wall thicknesses may be between 0.3 mm to 1.5 mm, for instance between 0.5 mm-1 mm in thickness.

Embodiments of spring carrier and associated apparatus/systems of the present disclosure are configured to securely retain a coil spring C therein, and reliably and accurately allow extraction of the coil spring C. In order that the coil spring can be both securely retained and accurately extracted, the spring carrier may be configured such that a certain clearance is provided between an outer diameter of the coil spring C and an inside wall of the inner cavity 13. The clearance may be set to allow substantially unimpeded insertion and extraction of the coil spring C into/from the inner cavity 13, yet also minimize lateral play or movement of the coil spring C within the inner cavity so that the coil spring can be accurately discharged where required. In an embodiment, such clearance may be 0.05 mm-0.3 mm, for instance between 0.1 mm-0.2 mm. Such clearance is shown as dimension d1 in FIG. 4A. In one embodiment, coil springs C to be received in the inner cavity 13 have a maximum outer diameter of 9.95 mm. Accordingly, an internal diameter d2 (shown in FIG. 4A) may be around 10.0 mm-12.95 mm, for instance around 10.05 mm-11.05 mm.

The embodiments of spring carrier 10 described herein comprise a flange 28. This may advantageously help in use of the spring carrier, such as with alignment as described above, or otherwise handling and manipulation of the spring carrier 10. However, the present disclosure is not intended to be limited to the spring carrier 10 having a flange 28 and in other embodiments, the flange 28 may be omitted.

Some embodiments of spring carrier 10 described herein operate with an electromagnet, either with the electromagnet 21 inducing a magnetic effect in a magnetic member 18 of the spring carrier, or the magnetic member 18 itself comprising an electro magnet. It is envisaged that the present disclosure may provide for such electromagnet embodiments to be able to generate a variable magnetic force, which may be controlled by varying the electrical power provided to the electro magnet. Such varying of the magnetic force may enable the spring carrier 10 to operate with coil springs of different sizes, mass or material. Apparatuses or systems of the present disclosure may comprise a controller connected or connectable to the electromagnet 21 or electromagnetic magnetic member 18 to control the magnetic force generated in use.

The embodiments of spring carrier 10 described herein comprise a magnetic member 18 to attract and retain a coil spring C within the inner cavity C of the hollow body 11. Within the scope of the present disclosure, the term magnetic element or magnetic member 18 is intended to encompass components which generate, or are capable of generating, a magnetic field for the magnetic attraction of a coil spring C made of a magnetically-attractable material. Such magnetic members 18 may comprise a single component or a mechanism or device, such as an electromagnet. Such magnetic members may include permanent magnets of any known material, components of a material which may be a magnetic material so as to be magnetisable, such as by electromagnetic induction, or generate a magnetic field, and components comprising an electromagnet. Such materials may include ferrous metals or materials incorporating ferrous metals by dispersion or otherwise provided in the make-up of the material. Such materials may include steel, iron, cobalt, or any other known magnetic or magnetisable material.

As is clear from the various different embodiments of the present disclosure described above, the spring carrier 10 may include an opening or aperture 38 at the second end 15 of the hollow body 11, or the second end may be entirely closed, such as by an end wall 17, a magnetic member 18, or an end wall 17 with a magnetic member 18 provided on the end wall 17. Embodiments in which the second end 15 of the hollow body 11 may not comprise an opening may comprise a magnetic member 18 provided externally of the hollow body 11, or embedded within the hollow body 11, as well as the magnetic member 18 being provided within the inner cavity 13.

The spring carrier 10 is shown and described, particularly in FIGS. 4A and 4B, as being configured such that the coil spring C is inserted into the spring carrier 10 while the spring carrier 10 is oriented with the first end 14 uppermost, so the coil spring C drops into the inner cavity 13 under gravity until the coil spring abuts the end wall 17 or/and magnetic member 18 at the second end 15 of the hollow body 11. However, use of the spring carrier 10 of the present disclosure is not limited to this use. Since the magnetic member 18 is capable of magnetically attracting the coil spring C such that the magnetic member 18 can support the weight of the coil spring C, the coil spring C may be inserted into the inner cavity 13 with the spring carrier 10 in any orientation.

It will be appreciated that the various embodiments of the present disclosure use a magnetic member 18 to attract and retain a coil spring C within the inner cavity 13. As such, the spring carrier 10 does not have any part which requires deflection or deformation in use of the spring carrier 10. This means that material fatigue and risk of damage in use is reduced or avoided, which may help extend the useful life of the spring carrier. This obviously has cost benefits as the spring carrier can be used for more operations in a manufacturing process before needing replacement, helping to reduce cost of manufacturing and thereby of the products being produced.

Some embodiments disclosed herein comprise a flange 28 extending around the perimeter of the hollow body 11 at the first proximal end 14 thereof. Such feature may optionally be applicable to all embodiments described herein. However, the disclosure is not intended to be limited to such feature and embodiments envisaged within the scope of the disclosure may not comprise a flange 28, or may comprise a flange disposed along the length of the hollow body other than at the remote end of the first proximal end, for example, at the second distal end 15, or intermediate the first proximal end and the second distal end.

Those of skill in the art will understand that modifications (additions and/or removals) of various components of the devices, apparatuses, methods, and embodiments described herein may be made without departing from the full scope and spirit of the present disclosure, which encompass such modifications and any and all equivalents thereof.

1-15. (canceled)

16. A spring carrier for receiving, retaining, and discharging of a coil spring in a manufacturing process, the spring carrier comprising:
an elongate hollow body defining an inner cavity configured to receive the coil spring;
an opening at a first proximal end of the hollow body for insertion of the coil spring into the inner cavity and extraction of the coil spring from the inner cavity; and
the hollow body including a second distal end opposite to the first proximal end, wherein the hollow body includes a magnetic member configured to magnetically attract and retain the coil spring when the coil spring is located within the inner cavity.

17. The spring carrier according to claim 16, wherein the magnetic member is provided proximate to the second distal end of the hollow body.

18. The spring carrier according to claim 17, wherein the magnetic member is disposed within the inner cavity.

19. The spring carrier according to claim 18, wherein the magnetic member comprises a permanent magnet.

20. The spring carrier according to claim 18, wherein the magnetic member comprises a magnetic material capable of induced magnetism.

21. The spring carrier according to claim 18, wherein the magnetic member comprises an electromagnet.

22. The spring carrier according to claim 16, wherein the magnetic member is disposed within the inner cavity.

23. The spring carrier according to claim 16, wherein the magnetic member comprises a permanent magnet.

24. The spring carrier according to claim 16, wherein the magnetic member comprises a magnetic material capable of induced magnetism.

25. The spring carrier according to claim 16, wherein the magnetic member comprises an electromagnet.

26. The spring carrier according to claim 16, wherein the hollow body includes a flange at the first proximal end of the hollow body and extending radially outwardly from the hollow body.

27. The spring carrier according to claim 16, comprising an aperture at the second distal end of the hollow body.

28. The spring carrier according to claim 27, wherein the hollow body comprises one or more protrusions extending inwardly at least partially across the aperture at the second distal end of the hollow body.

29. The spring carrier according to claim 16, comprising at least one window in a side wall of the hollow body to allow the coil spring located within the spring carrier to be visible from outside the spring carrier through the at least one window.

30. The spring carrier according to claim 16, wherein the opening at the first proximal end of the hollow body comprises a tapered region such that the opening widens towards the first proximal end.

31. An apparatus comprising:
a spring carrier comprising:
an elongate hollow body defining an inner cavity configured to receive a coil spring;
an opening at a first proximal end of the hollow body for insertion of the coil spring into the inner cavity and extraction of the coil spring from the inner cavity;
the hollow body including a second distal end opposite to the first proximal end,
wherein the hollow body includes a magnetic member configured to magnetically attract and retain the coil spring when the coil spring is located within the inner cavity; and
an electromagnet configured for placement proximate the spring carrier and configured to generate a magnetic field to make the magnetic member an induced magnet.

32. An assembly system comprising the apparatus of claim 31, the assembly system further comprising:
a coil spring manufacturing machine, wherein the coil spring manufacturing machine is configured to produce the coil spring, and
an insertion station arranged to feed the produced coil spring into the spring carrier.

33. An apparatus comprising:
a spring carrier comprising:
an elongate hollow body defining an inner cavity configured to receive a coil spring;
an opening at a first proximal end of the hollow body for insertion of the coil spring into the inner cavity and extraction of the coil spring from the inner cavity;
the hollow body including a second distal end opposite to the first proximal end,
wherein the hollow body includes a magnetic member configured to magnetically attract and retain the coil spring when the coil spring is located within the inner cavity; and
an airflow generator configured to generate a flow of air into the hollow body to facilitate extraction of the coil spring from the hollow body.

34. An assembly system comprising the apparatus of claim 33, the assembly system further comprising:
a coil spring manufacturing machine, wherein the coil spring manufacturing machine is configured to produce the coil spring, and
an insertion station arranged to feed the produced coil spring into the spring carrier.

35. A method of manipulating a coil spring using a spring carrier for receiving, retaining, and discharging of the coil spring in a manufacturing assembly process, the spring carrier comprising an elongate hollow body defining an inner cavity, an opening at a first proximal end of the hollow body, the hollow body including a second distal end opposite to the first proximal end, and a magnetic member provided proximate to the second distal end of the hollow body, the method comprising:
inserting the coil spring into the inner cavity through the opening at the first proximal end of the hollow body; and
magnetically attracting and retaining, with the magnetic member, the coil spring within the inner cavity.
